(19) United States
(12) Patent Application Publication
(10) Pub. No.: US 2004/0243219 A1
(43) Pub. Date: Dec. 2, 2004

(54) Venous prosthesis

(76) Inventors: Harald Fischer, Weingarten (DE); Bernd Vogel, Karlsruhe (DE)

Correspondence Address: KLAUS J. BACH & ASSOCIATES, PATENTS AND TRADEMARKS, 4407 TWIN OAKS DRIVE, MURRYSVILLE, PA, 15668, US

(21) Appl. No.: 10/883,176

(22) Filed: Jul. 1, 2004

Related U.S. Application Data
(63) Continuation-in-part of application No. PCT/EP03/01813, filed on Feb. 22, 2003.

(30) Foreign Application Priority Data
Feb. 26, 2002 (DE) 102 08 202.2

(51) Int. Cl. A61F 2/06

(52) U.S. Cl. 623/1.15; 623/1.24; 623/1.43

(57) ABSTRACT

In a venous prosthesis for a catheter-implantation into a blood vessel, a stent structure is provided which consists of a biocompatible material and which includes a unidirectional valve which consist of, or is coated with, a cell rejecting thrombose-inhibiting material.

This is a Continuation-In-Part Application of international application PCT/EP03/01813 filed Feb. 2, 2003 and claiming the priority of German application 102 08 202.2 filed Feb. 2, 2002.

### BACKGROUND OF THE INVENTION

The invention resides in a venous prosthesis for implantation in a blood vessel.

Tissue weaknesses in a human body, particularly in blood vessels, lead with increasing age to significant vessel expansion. This effect is particularly pronounced when the natural valve systems present in the circulation system in the human body have been stretched to such an extent that they do not function anymore as unidirectional valves that is as check valves, so that reflux characteristics occur which result in additional internal pressure exposure of certain blood vessels.

The effect results particularly in the formation of the so-called varicose veins in the legs. First, the blood vessels are expanded whereby also the body-internal vein valves in the hip area are stretched so that they finally fail. This results—aided by gravity forces—in a backup of the blood and further pressure loads in the veins of the legs. The resulting expansion or dilation of the veins first results in the formation of the varicose veins and, in an advanced stage, to the so-called open legs.

These symptoms can be treated by the use of support stockings which completely surround the legs so that a counter pressure against the blood pressure is established and the tissue in the leg is relieved. However, the body internal vein valves are not reconstituted in this way.

For a durable or prophylactic treatment of the varicose veins the maintenance or the re-establishment of the operability of the vein valves is very important. Since, upon determining the presence of varicose veins, it can be assumed that the tissue around the body internal vein valves has already been damaged, that is, dilated, the natural vein valves should be replaced or supported by the installation of a venous prosthesis that is an artificial vein valve.

U.S. Pat. No. 5,500,014 discloses a biological valve prosthesis consisting of a fluid valve and a support sleeve. The fluid valve which consists of a chemically fixed biological material is installed in that case at a certain desired location in a blood vessel and is fixed in position by the support sleeve surrounding the blood vessel. The support sleeve is shown as being a tubular component so that the installation is possible only by a major surgical procedure in which the blood vessel must be severed and inserted into the support sleeve.

It is the object of the present invention to provide a venous prosthesis which can be installed intravenously by means of a catheter and which does not require an additional support sleeve which extends around the blood vessel.

### SUMMARY OF THE INVENTION

In a venous prosthesis for a catheter-implantation into a blood vessel a stent structure is provided which consists of a biocompatible material and which includes a unidirectional valve, which consist of, or is coated with, a cell rejecting thrombose-inhibiting material.

The main feature of the invention resides in the integration of a strut structure of a bio-compatible material which is capable of growing together with the blood vessel where it is in contact with the blood vessel. However, the interior lumen of the stent structure remains free of any growth to permit blood to flow therethrough. In this way, the venous prosthesis is permanently fixed in the blood vessel in a particularly advantageous manner without separate parts. In addition, the tissue is stabilized by the stent structure so as to prevent undesirable dilation.

The stent structure includes a unidirectional fluid valve. In order to avoid that the fluid valve grows together with the tissue of the blood vessel or other body tissue, the valve consists of a cell rejection material or it is coated by such a material. Such materials are, for example, plasma-polymerized polyethylene glycols (hydro-gels).

For the insertion of the venous prosthesis into a blood vessel the venous prosthesis is first radially elastically compressed and inserted into a catheter. The surgery is performed in a particularly patient-accommodating minimally invasive (endo-luminal) manner, wherein, in a first step, the catheter is inserted into the blood vessel at a location close to the position selected for the application of the venous prosthesis. When the position is reached by the distal end of the catheter, the venous prosthesis is pushed out of the catheter. Then the radially elastic compression of the venous prosthesis is eliminated by the super-elastic properties of the material whereby the stent structure expands radially and engages the wall of the blood vessel. The outer diameter of the expanded stent structure is slightly larger than the inside diameter of the blood vessel so that a slight radial outward pressure against the blood vessel is established and the venous prosthesis is fixed thereby in the desired position in the blood vessel.

As materials for the stent structure shape memory or hyper-elastic materials, which additionally must be biocompatible, are particularly suitable. Another quite suitable group of materials are the metallic shape memory alloys, especially NiTi alloys which have highly super-elastic properties and which can be worked in the sub-millimeter range for example by laser or erosion procedures. Other suitable shape memory materials with the required properties are shape memory polymers or super-elastic copper alloys which have good biocompatible properties and also elastic properties. Typical representatives of the mentioned group of hyper-elastic biocompatible materials suitable for the purpose are hyper-elastic polymers (for example, Elasteon) or single-crystal copper alloys.

The stent structure with the fluid valve must also have such a geometric shape that it can be compressed elastically in radial direction. It is proposed to provide the stent structure in form of a sieve-like tube member, wherein all the openings in the tube wall have the same shape, for example, that of a rhombus, and the longer diagonals of the rhombus-like opening extend in axial direction. The webs between adjacent openings which therefore have an angle of less than 45° with respect to the axis of the tube serve as bending spring elements providing for the possible radially elastic compression of the stent structure.

The fluid valve must have a surface which rejects cells so that it cannot grow together with the tissue of the blood vessel nor with any other body tissue components. It is proposed to prepare the fluid valve either as a separate component of a cell-rejecting material or, if the fluid valve is made as part of the stent structure from the same tube member by erosion or laser manufacturing procedures, to coat the valve with a cell rejecting material.

The fluid valve must be a unidirectional valve for which it is not necessary to provide a perfect seal in one direction and an unrestricted fluid flow in the opposite direction. Rather a flow capability in both directions is acceptable for the use of the valve as a venous prosthesis if the flow resistance in the backward direction is significantly larger than the flow resistance in the forward direction. For the purpose of a venous prosthesis, it is sufficient if the fluid passage is only essentially open or essentially closed by the fluid valve.

In order to avoid rejection reactions by the use of indications, it is possible to make the venous prosthesis or parts thereof from a polymer, which contains medication or which is permeable to medications or to coat it or parts thereof with such a material.

Below, several embodiments of the venous prosthesis according to the invention will be described in greater detail on the basis of the accompanying drawings.

### BRIEF DESCRIPTION OF THE DRAWINGS

FIGS. 1a to 1c show exemplary embodiments of venous prostheses with stent structures and separate fluid valves installed in the stent structures, and

FIGS. 2a to 2c show exemplary venous prostheses wherein the stent structures and the fluid valves are formed integrally from one compound.

### DESCRIPTION OF THE PREFERRED EMBODIMENTS

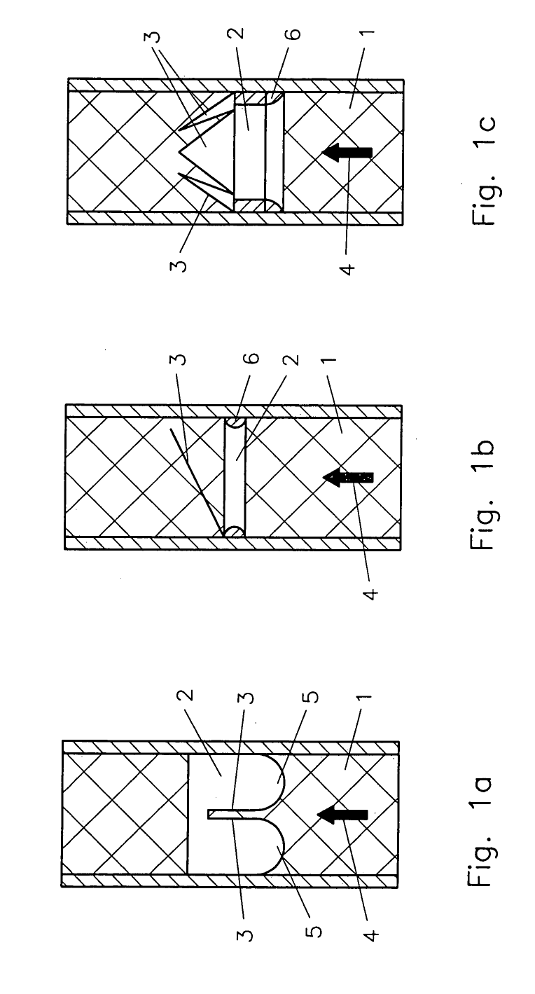

FIGS. 1a to 1c show different embodiments of the invention each comprising a stent structure 1 with a fluid valve 2 installed in the stent structure 1. In this case, the fluid valve may be of a different, that is, a cell-rejecting material preferably a corresponding plastic material. The fluid valve 2 may be connected to the stent structure by casting, melting, cementing or another suitable procedure. For the manufacture of the fluid valve as a plastic material part, an injection molding- or micro-casting process may be used. Alternatively, the fluid valve may consist of a metallic material, preferably a nickel-titanium alloy and may be attached to the stent structure by welding, soldering or cementing.

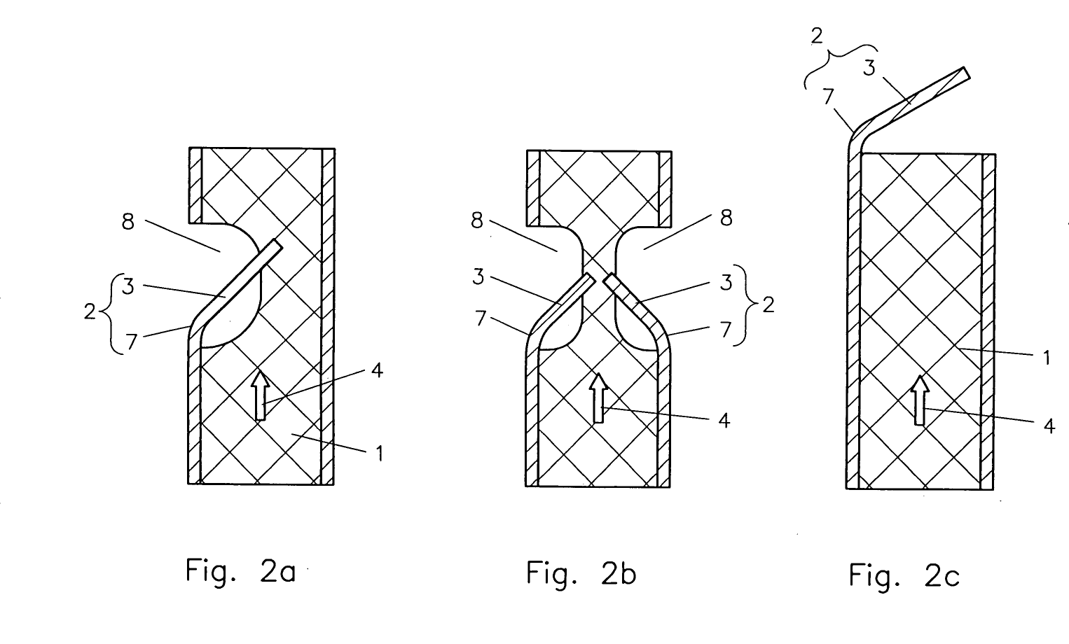

In all FIGS. 1a to 1c and also in FIGS. 2a to 2c, the flow direction of the respective unidirectional valves 2 is indicated by an arrow 4.

FIG. 1a shows a fluid valve 2 with two flaps 3, which are bendable and which open radially or close depending on the flow direction of the fluid whereby the flow resistance is significantly reduced or, respectively, increased—depending on the flow direction. The flaps must in this case be bendable and not necessarily elastic. The effect of the unidirectional fluid valve is improved if the bending resistance of the curved areas 5 in contact with each other becomes smaller with a progressing rolling along each other in the direction of the arrow 4.

Basically, the stent structure for a flap valve 3 of polymer may be formed from a thin wire of a shape memory alloy.

FIGS. 1b and 1c show each a fluid valve with one flap (FIG. 1b) or, respectively, with several flaps 3 (FIG. 1c), which are connected to a valve seat 6 by pivot joints or by joint-free connections consisting of a shape memory alloy. In order to achieve the necessary radially elastic compressibility of the venous prosthesis, the flaps 3 as well as the valve seals 6 must have sufficient flexibility. Particularly the flap 3 of the embodiment according to FIG. 1b must have sufficient elastic bending capability.

FIGS. 2a to 2c show other embodiments wherein the stent structure 1 and the fluid valves 2 are formed integrally from an unfinished body. The fluid valves 2 have a single flap (FIGS. 2a and 2c) or two flaps 3 (FIG. 2b) may be provided. Each flap 3 is connected to the stent body by way of an elastic bending structure 7, which is formed as part of the stent body. FIGS. 2a and 2b further show cutouts 8 formed in the stent body 1 which extend over an area as needed to form the flaps 3 from the respective structures.

The venous prosthesis according to FIGS. 2a to 2c may consist of a plastic material and be manufactured by injection molding, casting or micro-casting procedures or they may be manufactured from a shape memory alloy in the form of a tubular raw material by erosion or laser cutting procedures. The cutouts 8 are formed by utilizing the respective areas to form the flaps 3. Preferably, the venous prosthesis consists of a bio-compatible material and the fluid valves 2 are coated by a cell rejecting material.

In principle, the base material for the manufacture of the stent structure may be a wire material (shape memory material or hyper-elastic material) if the wire structure is welded, cemented or otherwise joined at the contact areas.

What is claimed is:

1. A venous prosthesis for a catheter-implantation into a blood vessel, comprising:
a) a stent structure (1) of a biocompatible material which is implanted into the blood vessel for growing together with the blood vessel, and
b) a unidirectional fluid valve (2) firmly installed in the stent structure (1), said fluid valve consisting at least at the surface thereof of a cell rejecting thrombose-inhibiting material.

2. A venous prosthesis according to claim 1, wherein said stent structure (1) consists of one of a shape memory material and a hyper-elastic material.

3. A venous prosthesis according to claim 1, wherein said stent structure (1) consists of a nickel titanium alloy.

4. A venous prosthesis according to claim 1, wherein said fluid valve (2) comprises at least one flap (3).

5. A venous prosthesis according to claim 4, wherein said at least one flap (3) of said fluid valve (2) is an integral part of said stent structure (1).

6. A venous prosthesis according to claim 1, wherein said fluid valve (2) is a plastic part which is molded or cast together with the stent structure.

7. A venous prosthesis according to claim 1, wherein said fluid valve (2) consists of a nickel titanium alloy and is fixed in the stent structure (1) by one of welding, soldering and cementing.

8. A venous prosthesis according to claim 1, wherein said fluid valve (2) and said stent structure are manufactured from a single part.

9. A venous prosthesis according to claim 1, wherein at least parts of said venous prosthesis consist at least at the surfaces thereof of a medication containing or medication permeable material.
